(19) United States
(12) Patent Application Publication
(10) Pub. No.: US 2024/0358253 A1
(43) Pub. Date: Oct. 31, 2024

(54) DIGITAL TESTING SYSTEM, METHOD AND APPLICATION FOR COLOR VISION DEFICIENCY

(71) Applicants: Shakeb Siddiqui, Springfield, NJ (US); Connor Sheehan, Erie, PA (US)

(72) Inventors: Shakeb Siddiqui, Springfield, NJ (US); Connor Sheehan, Erie, PA (US)

(21) Appl. No.: 18/440,152

(22) Filed: Feb. 13, 2024

Related U.S. Application Data
(60) Provisional application No. 63/461,682, filed on Apr. 25, 2023.

(51) Int. Cl. A61B 3/06; G06F 8/61; G16H 10/60; G16H 50/20

(52) U.S. Cl. CPC A61B 3/066; G06F 8/61; G16H 10/60; G16H 50/20

(57) ABSTRACT

A digital color vision deficiency testing system is disclosed. The system includes an application for providing a plurality of user interfaces for performing color vision testing. A user interface provides, for example, a color matrix having a central color matrix, including a plurality of color combinations, an inner matrix, and an outer matrix. For testing, a user selects a color and, for example selects an interactive marker (i.e., finger, stylus, or mouse) across the central color matrix to dynamically change the color of the inner matrix to match with the color of the outer matrix, wherein the RGB, RGBA, HEX, HSV, HSL, YUV/YCBCR, CIELAB, HSLA, and/or CMYK values are recorded when the user lifts the marker or clicks the mouse (i.e., stops movement). A plurality of iterations is performed and then, the recorded values are compared with stored threshold values for providing an accuracy result to indicate color vision deficiency.

### CROSS-REFERENCE TO RELATED APPLICATION

The present application claims priority to, and the benefit of, U.S. Provisional Application No. 63/461,682, which was filed on Apr. 25, 2023 and is incorporated herein by reference in its entirety.

### FIELD OF THE INVENTION

The present invention generally relates to the field of color blindness testing devices and methods. More specifically, the present invention relates to a novel digital color vision testing system that provides a mobile application and a website for performing online color vision deficiency testing. A color matrix (i.e., color wheels, color images, color boxes) is displayed on an interface that enables a user to select an interactive marker (i.e., a finger, a stylus, or a mouse) to match a color of an inner matrix with an outer matrix to record different RGB, RGBA, HEX, HSV, HSL, YUV/YCBCR, CIELAB, HSLA, or CMYK values. The recorded values are compared with the threshold values to provide deficiency results. Accordingly, the present disclosure makes specific reference thereto. Nonetheless, it is to be appreciated that aspects of the present invention are also equally applicable to other like applications, devices, and methods of manufacture.

### BACKGROUND

By way of background, color vision deficiency, or color blindness, is a condition that causes inability or difficulty in perceiving certain colors. Commonly, color blindness is more prevalent among males, affecting approximately 8% of men of Northern European descent, while 0.5% females are affected by the color blindness. Many individuals with color vision deficiency may become aware of their condition later in life due to various reasons. One contributing factor is the lack of accessibility and limitations of current color vision testing methods.

Traditional tests, such as the Ishihara test, have been widely used for detecting color blindness. These tests typically involve physical books containing plates with numbers or patterns embedded in dots of various colors. Further, the Ishihara test and similar conventional tests are usually conducted during visits to an optometrist or a medical doctor. However, these tests have some limitations. Firstly, they provide a limited diagnosis, indicating only whether a person is colorblind or not, without providing detailed information about the extent or specific nature of their color vision deficiency. Present conventional testing is limited to colorblind categories of red-green, blue-yellow, and monochromy. These outcomes fail to capture the full spectrum of color vision capabilities. Furthermore, the traditional color vision tests are not always easily accessible or convenient for many individuals. The physical books and specialized equipment used in these tests can be restricted to certain medical settings, making them less accessible for individuals who may want to evaluate their color vision outside of a clinical environment. Also, factors such as lighting conditions, the age of the test book, and the interpretive skills of the person administering the test can also impact the correctness of the test.

Conventional tests also do not account for the advancements in digital displays and the increasing dependence on digital interfaces in our daily lives. With the rise of digital technology, color vision plays a crucial role not only in real-world interactions but also in our virtual experiences. Yet, traditional color vision tests remain ill-equipped to assess and diagnose color vision deficiencies in the context of these digital platforms. Also, conventional tests are not customized to individual patients and thus are not effective. People desire an improved color blindness test that overcomes the problems associated with the traditional color vision tests.

Therefore, there exists a long felt need in the art for a modern, comprehensive, and easily accessible color vision test. There is also a long felt need in the art for a color vision test that includes the advancements in digital display technologies. Additionally, there is a long felt need in the art for a color vision test system that can be used by people sitting at home without requiring a visit to an optometrist or a doctor. Moreover, there is a long felt need in the art for an improved color vision test system that does not give a binary result of color vision test but provides a nuanced understanding of an individual's color vision abilities. Further, there is a long felt need in the art of an improved color vision test system that does not use physical books and can be accessed on digital devices. Furthermore, there is a long felt need in the art for an easily accessible color vision test that allows individuals to detect color vision defects early. Finally, there is a long felt need in the art for a color vision test system that provides detailed information about an individual's color vision capabilities across the entire spectrum.

The subject matter disclosed and claimed herein, in one embodiment thereof, comprises a computer implemented method for testing color vision deficiency of users. The method comprising the steps of providing a mobile application or a website configured to provide an interactive Red, Green, Blue (RGB) color matrix (i.e., color wheels, color images, color boxes), the RGB color matrix (i.e., color wheels, color images, color boxes) includes a central RGB color matrix, an inner matrix, and an outer matrix, the inner matrix circumscribes the central color matrix and the outer matrix circumscribes the inner matrix. The central RGB color matrix displays a plurality of color combinations, and the method receives, from a user, a plurality of RGB, RGBA, HEX, HSV, HSL, YUV/YCBCR, CIELAB, HSLA, or CMYK values when the user selects an interactive marker (i.e., a finger, a stylus, or a mouse) across the central color matrix to dynamically adjust the color of the inner matrix to match the color of the outer matrix wherein the received RGB, RGBA, HEX, HSV, HSL, YUV/YCBCR, CIELAB, HSLA, or CMYK values are recorded when the user lifts the marker (i.e., finger or stylus) or clicks the mouse; comparing, the received RGB, RGBA, HEX, HSV, HSL, YUV/YCBCR, CIELAB, HSLA, or CMYK values with a plurality of stored threshold RGB, RGBA, HEX, HSV, HSL, YUV/YCBCR, CIELAB, HSLA, or CMYK values, and displaying accuracy result based on comparison of the received RGB, RGBA, HEX, HSV, HSL, YUV/YCBCR, CIELAB, HSLA, or CMYK values with the stored threshold RGB, RGBA, HEX, HSV, HSL, YUV/YCBCR, CIELAB, HSLA, or CMYK values wherein, the result includes individual and separate accuracy results for Red color, Green color, and Blue color.

In this manner, the color vision testing system and associated mobile application and website of the present invention accomplish all of the forgoing objectives and provide users with a digital platform that provides an online color vision test, replacing traditional tests conducted using physical books. The test does not categorize individuals into predefined groups and provides the color vision deficiency across the wide spectrum of color vision abilities. The system offers detailed assessments of individual color vision capabilities and can be used in different settings by users.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises computer implemented color vision test system. The system further comprising a software application and a website accessible using an electronic device, wherein the application provides different user interfaces for testing color vision using the electronic device, a server system including an application server configured to provide the plurality of user interfaces for testing color vision via the application and the website, a statistical model or machine learning model can be used for interpreting test results and for providing personalized and interactive support to users, a clinical information database for storing color vision threshold data for diagnosis, including FDA approval information for color vision identification across the spectrum, and a user information database for storing user information, diagnosis data, and user feedback on test results.

In yet another embodiment, a computer implemented method for testing color vision deficiency of users is described. The method comprising the steps of providing a user interface in a mobile application or on a website, the user interface displays an interactive Red, Green, Blue (RGB) color matrix, the RGB color matrix comprising a central RGB color matrix, an inner matrix, and an outer matrix, the inner matrix circumscribes the central color matrix and the outer matrix circumscribes the inner matrix. The central RGB color matrix displays multiple color combinations, and the method receives, from a user, at least one RGB value when the user selects the marker (i.e., a finger, a stylus, or a mouse) across the central color matrix to dynamically adjust the color of the inner matrix to match the color of the outer matrix wherein the received RGB values is recorded when the user lifts the marker or clicks the mouse, comparing, the received RGB values with a plurality of stored threshold RGB values, and displaying accuracy result based on comparison of the received RGB values with the stored threshold RGB values wherein, the result includes individual and separate accuracy results for Red color, Green color, and Blue color.

In yet another embodiment, a plurality of RGB values is received from the user, wherein the user lifts the marker or clicks the mouse when the color of the inner matrix matches with the color of the outer matrix.

In yet another embodiment, a plurality of RGB, RGBA, HEX, HSV, HSL, YUV/YCBCR, CIELAB, HSLA, or CMYK values are received from the user and are compared to the threshold RGB, RGBA, HEX, HSV, HSL, YUV/YCBCR, CIELAB, HSLA, or CMYK values for displaying the accuracy result.

In yet another embodiment, the plurality of RGB, RGBA, HEX, HSV, HSL, YUV/YCBCR, CIELAB, HSLA, or CMYK values, or any other digital representations of color (i.e., color representation models or digital color encoding systems), are received from the user when the user lifts the marker or clicks the mouse upon matching of the color of the inner matrix with the color of the outer matrix.

In yet another embodiment, machine learning and statistical models provide interpretation of accuracy results and are displayed on a user interface, wherein one or more queries can be received from the user for generating responses.

In a further embodiment, the color vision test system uses a statistical or machine learning model for using algorithms and statistical models for calculating accuracy results and interpretation, wherein the statistical or machine learning model uses threshold values and feedback from users for providing an accurate score and interpretation.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of a color vision test system of the present invention in accordance with the disclosed architecture;

FIG. 2 illustrates the home page displayed by the website for enabling users to take a color vision test in accordance with the disclosed architecture;

FIG. 3 illustrates a prompt displayed by the website before starting the color vision test of users in accordance with the disclosed architecture;

FIG. 4 illustrates an exemplary user interface displayed to a user for performing color vision test in accordance with the disclosed architecture;

FIG. 5 illustrated a test result user interface displayed to a user upon completion of the color vision test in accordance with the disclosed architecture;

FIG. 6 illustrates a platform enabled user interface offered by the system for assisting users in interpreting their test results in accordance with the disclosed architecture;

FIG. 7 illustrates a flow chart depicting a process of training of a module of the ‘TrueHue’ system of the present invention in accordance with the disclosed architecture;

FIG. 8 illustrates a flow chart depicting a process of use of the ‘TrueHue’ system of the present invention by optometrists for understanding a patient's color vision abilities in accordance with the disclosed architecture; and

FIG. 9 illustrates a flow chart depicting a process of evaluating color vision test data collected from a user for evaluating RGB score in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a modern, comprehensive, and easily accessible color vision test. There is also a long felt need in the art for a color vision test that includes the advancements in digital display technologies. Additionally, there is a long felt need in the art for a color vision test system that can be used by people sitting at home without requiring a visit to an optometrist or a doctor. Moreover, there is a long felt need in the art for an improved color vision test system that does not give a binary result of color vision test but provides a nuanced understanding of an individual's color vision abilities. Further, there is a long felt need in the art of an improved color vision test system that does not use physical books and can be accessed on digital devices. Furthermore, there is a long felt need in the art for an easily accessible color vision test that allows individuals to detect color vision defects early. Finally, there is a long felt need in the art for a color vision test system that provides detailed information about an individual's color vision capabilities across the entire spectrum.

The present invention, in one exemplary embodiment, is a computer implemented color vision test system. The system includes a software application and a website accessible using an electronic device, wherein the application provides different user interfaces for testing color vision using the electronic device, a server system including an application server configured to provide the plurality of user interfaces for testing color vision via the application and the website, interpreting test results, and providing personalized and interactive support to users. A clinical information database can be provided for storing color vision threshold data for diagnosis, including FDA approval information for color vision identification across the spectrum, and a user information database for storing user information, diagnosis data, and user feedback on test results.

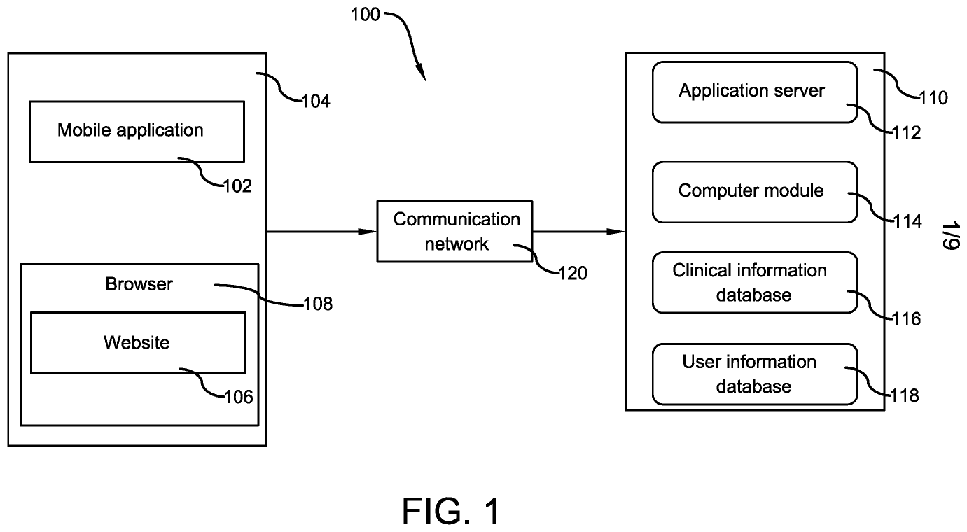

Referring initially to the drawings, FIG. 1 illustrates a schematic view of a color vision test system of the present invention in accordance with the disclosed architecture. The color vision test system 100 of the present invention is designed to provide improved color vision test functionality to users by facilitating accurate identification of potential color vision deficiencies. More specifically, the system 100 includes a software/application 102 accessible using an electronic device or mobile device 104. The application 102 can be downloaded and installed in the electronic device 104. Similarly, functionalities offered by the system 100 can be accessed using a website 106 accessible using a browser 108 via a uniform resource location (URL). The application 102 and the website 106 are configured to provide similar user interfaces for allowing users to test their color vision using mobile devices 104.

The system 100 has a server system 110 that includes an application server 112 configured for providing different user interfaces for enabling users to test their color vision using the application 102 and the website 106. A statistical or machine learning model 114 is integrated in the server system 110 and helps users of the system 100 in interpreting test results for a better understanding of their color vision capabilities. The statistical or machine learning model 114 is also programmed to provide responses to queries asked by the users of the system 100 for providing personalized and interactive support to the users.

A clinical information database 116 is designed to store color vision threshold data used for providing diagnosis to users. The clinical information database 116 is used for providing clinical diagnosis to users and can also include FDA approval information for correct identification of the color vision across the spectrum. A user information database 118 is used for storing user information database. The database 118 can store diagnosis data of users and their feedback on the results. In some embodiments, the database 118 can also store personal information such as name, age, geography, gender, earlier color vision defects, and more. The system 100 uses the information stored in the clinical information database 116 and the user information database 118 for providing an accurate color vision identification of users.

The server system 110 can be accessed by users using a communication network 120 which can be a public network such as the Internet or can also be a private network, depending on implementation of the color vision test system 100.

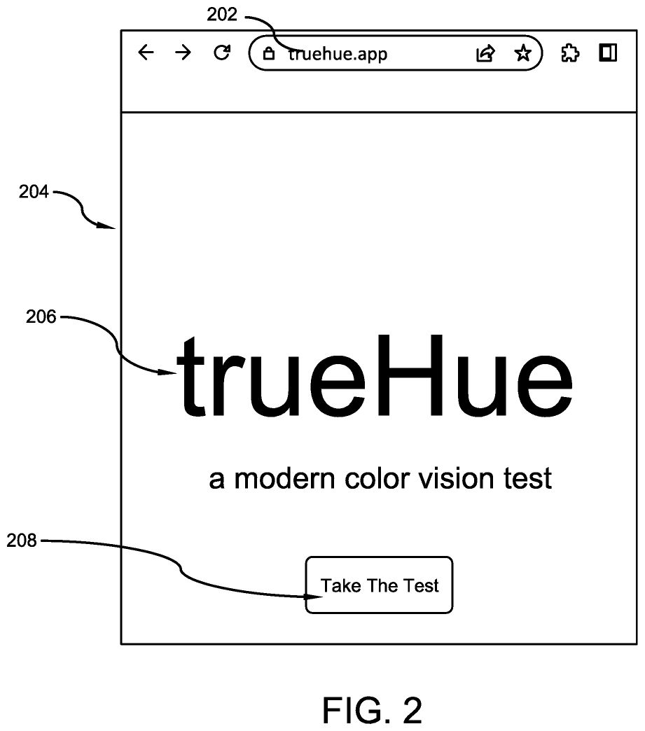

FIG. 2 illustrates the home page displayed by the website 106 for enabling users to take a color vision test in accordance with the disclosed architecture. In the present embodiment, the website 106 is shown as an example but the same interface can also be displayed by the application 102. Upon accessing the website 106 using the URL 202, the home page 204 is displayed which includes the logo 206 and a “Take The Test” icon 208 which can be activated by touching thereon for starting the color vision test.

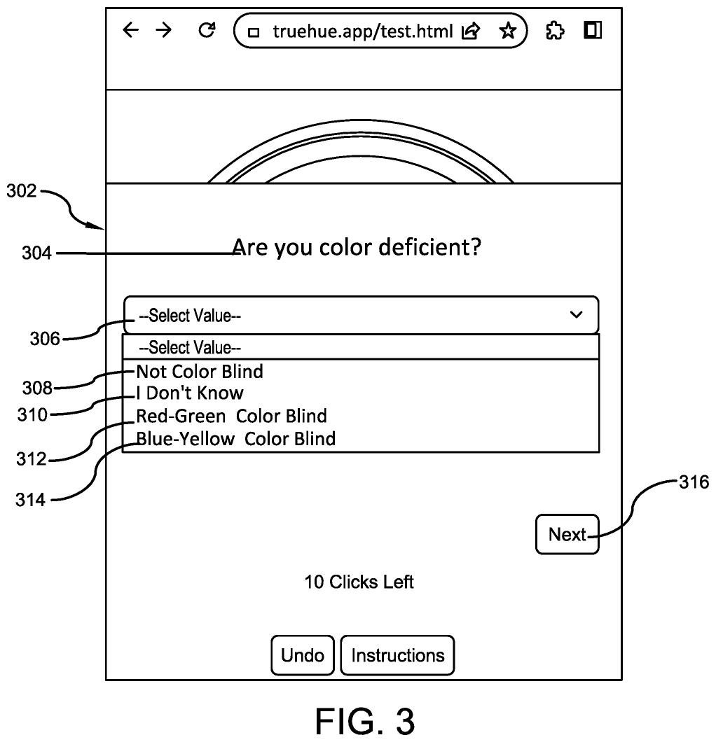

FIG. 3 illustrates a prompt displayed by, for example, the website 106 before starting the color vision test of users in accordance with the disclosed architecture. Upon activating “Take The Test” icon 208 as described in FIG. 2, the user interface 302 is displayed which includes an “Are you color deficient?” prompt 304 for enabling a user to input to the system 100 information about current status of the color vision defect. The prompt 304 provides a drop-down menu 306 that includes options “Not Color Blind” 308, “I Don't Know” 310, “Red-Green Color Blind” 312 and “Blue-Yellow Color Blind” 314. A user of the website 106 can choose any of the drop-down menu options and accordingly, the system 100 provides a customized color vision test. The customized color vision test is displayed on clicking the “Next” button 316 on the interface 302.

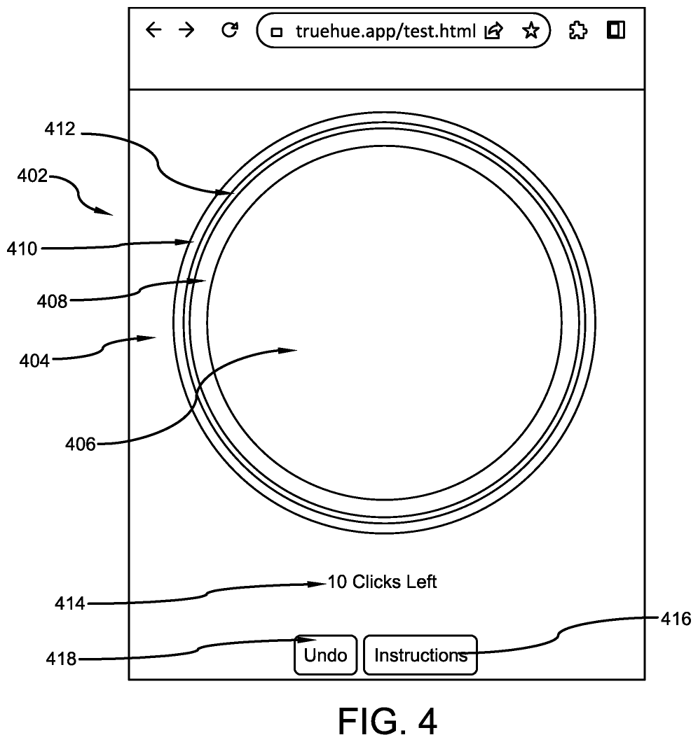

FIG. 4 illustrates an exemplary user interface displayed to a user for performing color vision test in accordance with the disclosed architecture. The color vision test system 100 provides the intuitive ‘TrueHue’ user interface 402 that centers around an interactive Red, Green, Blue (RGB) color matrix 404. The RGB color matrix 404 facilitates the collection of in-depth data from a user for testing color vision. The collected in-depth data is essential for accurately identifying potential color vision deficiencies. The RGB color matrix 404 includes a central RGB color matrix 406, an inner matrix 408 and an outer matrix 410. A black partition 412 separates the inner matrix 408 and the outer matrix 410. The central RGB color matrix 406 includes a plurality of color combinations and for testing color vision, the color of the inner matrix 408 is required to match with the outer matrix 410. During color vision test, a user selects their marker (i.e., a finger, a stylus, or a mouse) across the central color matrix 406 such that the color of the inner matrix 408 dynamically adjusts based on the position of the user's cursor or touch. Upon matching color of the inner matrix 408 and the outer matrix 410, the user can lift up their marker or click the mouse to select the color.

It should be noted that the colors displayed in the central RGB color matrix 406 can depend on the type of display interface and can also depend on the drop-down menu options as described in FIG. 3. A predetermined number of rounds, preferably ten or more (shown as “10 Clicks left” 414), are performed during color vision test wherein, in each round, the user matches the color of the inner matrix 408 and the color of the outer matrix 410. In the preferred embodiment, the color of the outer matrix 410 remains static and color of the inner matrix 408 changes dynamically upon movement of, for example, the marker (i.e., a finger, a stylus, or a mouse) along the central wheel 406. Upon completion of the rounds, all RGB data points are collected by the application 102 website 106 and are securely transmitted to the server system 110 and stored in the clinical information database 116 for further analysis. The user interface 402 provides the options of reading instructions of performing the test using the “Instructions” button 416 and any round can be undone by the user using the “Undo” tab 418.

The color vision test system 100, during the color vision testing process, collects an extensive range of RGB data. During each round, ‘TrueHue’ system 100 records the RGB color values that are shown to the user in the outer matrix 410 and the inner matrix 408, as well as the corresponding color values selected by the user while moving, for example, the finger/stylus/mouse along the central wheel 406. The collected data is used by the system 100 to calculate the deviation between the displayed and chosen colors, which is a fundamental metric in assessing color vision proficiency of the user.

The system 100 also uses multidimensional data points for constructing an individual's color vision capabilities and potential deficiencies. A pattern of high deviations across different rounds indicates potential color vision deficiencies. For identifying high deviations, the system 100 also tracks the time taken by the user to match each color in each round. The more time taken by the user for matching the colors indicates challenges with color differentiation which is a common symptom of color vision deficiency. The ‘TrueHue’ system 100 also monitors and records the path the user navigates across the color matrix 406 to find the matching color. This traversal data offers vital insights into the user's color searching strategy and their spatial color perception skills. The path a user navigates around the color matrix 406, including any hesitation or back-and-forth movements, provides additional indications of color vision challenges.

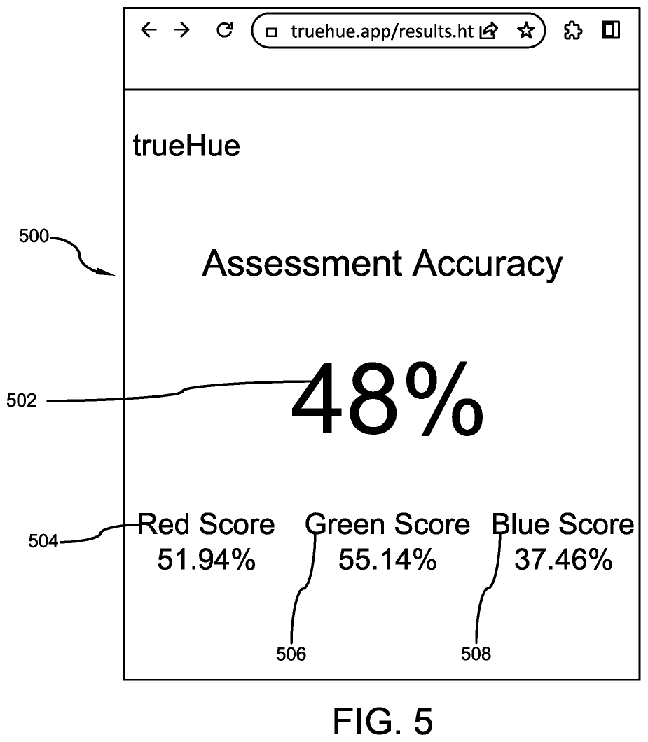

FIG. 5 illustrates a test result user interface displayed to a user upon completion of the color vision test in accordance with the disclosed architecture. Upon completion of the color vision test, the TruHue test system 100 provides the test result user interface 500 that displays a score sheet reflecting the overall accuracy of their color matching attempts. The total score 502 is represented as a percentage and summarizes a user's performance across all rounds. Also, the user interface 500 displays a Red score 504, a Green score 506, and a Blue score 508 for providing specific strengths and weaknesses across the RGB spectrum. A high score indicates a correct vision with less chances of color vision defect. As described earlier, for calculating the scores, multidimensional data points are used and the scores are based on the individual's color vision capabilities and potential deficiencies.

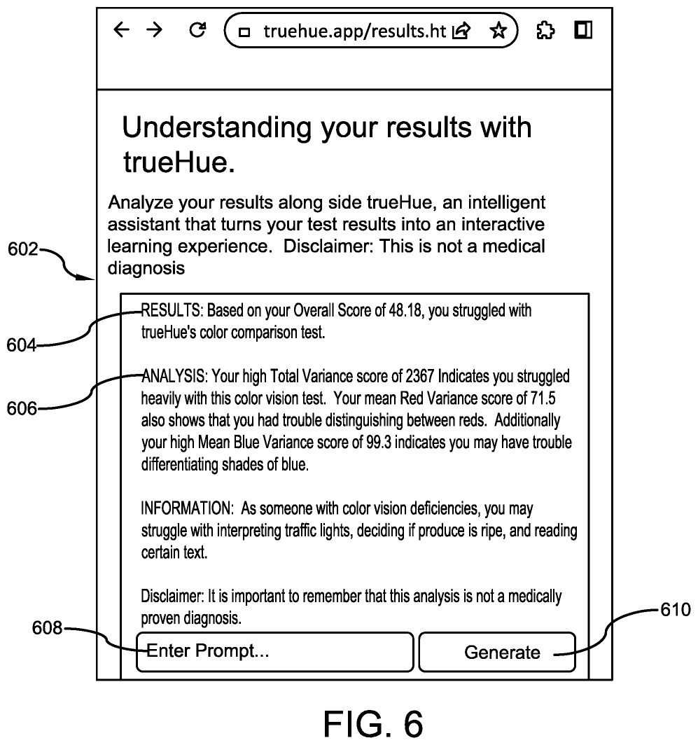

FIG. 6 illustrates a platform enabled user interface offered by the system 100 for assisting users in interpreting their test results in accordance with the disclosed architecture. The ‘TrueHue’ system 100 includes the statistical or machine learning model 114 which is programmed to receive user queries and interpret and respond for providing responses to the user queries. As illustrated, the computer interface 602 provides a response box 604 which provides a system generated interpretation 606 of the scores 502, 504, 506, 508 described in FIG. 5.

The computer interface 602 also enables users to input a prompt such as a question using the prompt box 608 and generate response using “Generate” option 610. The statistical or machine learning model 114 and the computer interface 602 can be based on any proprietary technology or can also be based on an open platform. Further, the statistical or machine learning model 114 is responds in real-time based on the color vision tests conducted by the system 100. The computer interface 602 offers personalized, interactive support, ensuring that users are well-informed and confident in their understanding of their results and the implications for their color vision abilities.

In some embodiments of the present invention, the statistical or machine learning model 114 also uses FDA approval data along with the information received from the users, thereby helping to provide clinical diagnosis of color vision deficiencies.

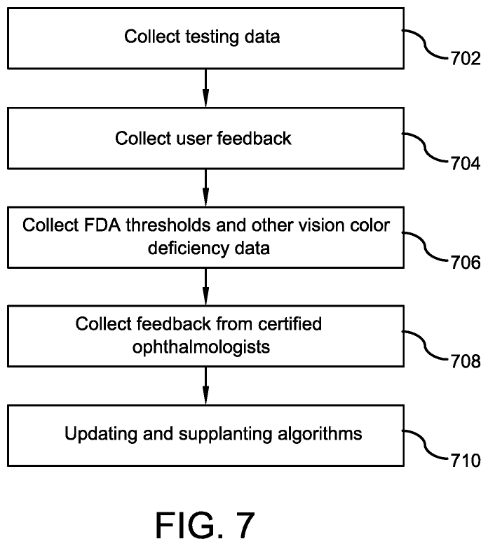

FIG. 7 illustrates a flow chart depicting a process of training of the statistical or machine learning model 114 of the ‘TrueHue’ system of the present invention in accordance with the disclosed architecture. Initially, real-world data from analysis of user color vision deficiency testing of users by the system 100 is stored in the statistical or machine learning model 114 for updating and supplanting the algorithms (i.e., statistical models) included in the statistical or machine learning model (Step 702). Then, user feedback on the scores calculated by the system 100 is stored in the statistical or machine learning model for supervised learning (Step 704). Thereafter, FDA thresholds and other vision color deficiency data are stored in the statistical or machine learning model (Step 706). The FDA data can be automatically included in the statistical or machine learning model from third party databases.

Finally, feedback on RGB values used in the color matrix from certified ophthalmologist are collected and used in the statistical or machine learning model for learning (Step 708). Using the data stored in the above steps, the algorithms and statistical models included in the statistical or machine learning model 114 are updated and supplanted in real time for providing accurate color deficiency information across RGB spectrum (Step 710).

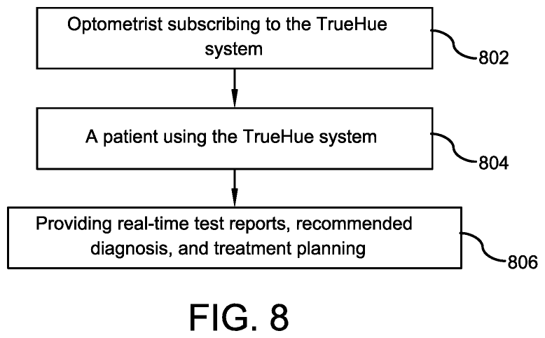

FIG. 8 illustrates a flow chart depicting a process of use of the ‘TrueHue’ system of the present invention by optometrists for understanding a patient's color vision abilities in accordance with the disclosed architecture. Initially, an optometrist subscribes to the ‘TrueHue’ system for accessing tests offered by the system (Step 802). In preferred embodiment, different user interfaces and testing functionality offered by the system can be accessed on any conventional display screen but in some embodiments, a special computing device having a touch-based or non-touch based display screen can also be used for accessing offerings of the system 100.

Then, a patient uses the ‘TrueHue’ system 100 at the premises of the optometrist for performing a color vision test (Step 804). Once the test is complete, the optometrist is provided with real-time test reports, recommended diagnosis, and treatment planning (Step 806). The subscription-based model helps in providing a sustainable revenue stream for ‘TrueHue’ system 100 while providing ongoing value and support to optometrists' office.

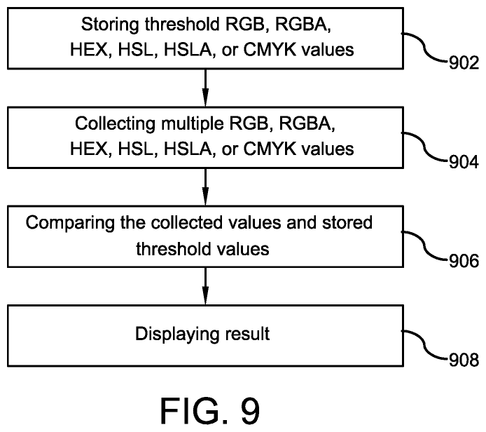

FIG. 9 illustrates a flow chart depicting a process of evaluating color vision test data collected from a user for evaluating RGB score in accordance with the disclosed architecture. For evaluation of color vision deficiency of a user, the threshold RGB, RGBA (A: Alpha is opacity level of color), HEX (hexadecimal values), HSL (hue-saturation-lightness), HSLA (huc-saturation-lightness-alpha), or CMYK (Cyan-Magenta-Yellow-Black) values are stored in the statistical or machine learning model (Step 902).

Then, in step 904, multiple RGB, RGBA, HEX, HSV, HSL, YUV/YCBCR, CIELAB, HSLA, or CMYK values, or any other digital representations of color, are collected across multiple rounds from a user (Step 904). Then, the server system 110 compares the stored threshold values with the collected values (Step 906) and based on the comparison, results are displayed (Step 908) as illustrated in FIGS. 5 and 6.

It should be noted that the ‘TrueHue’ system 100 provides a digital platform that provides an online color vision test, replacing traditional tests conducted using physical books. The system 100 recognizes color vision as a spectrum and offers detailed assessments of individual color vision capabilities. Further, the system 100 can be used in different settings such as optometrists office, in health check-ups and can also be used as an education tool for developing a deep level of understanding of the color vision tests.

Embodiments of the present disclosure take the form of computer-executable instructions, including algorithms and statistical models executed by a programmable computer. However, the disclosure can be practiced with other computer system configurations as well. Certain aspects of the disclosure can be embodied in a special-purpose computer or data processor that is specifically programmed, configured, or constructed to perform one or more of the computer-executable algorithms described below. Accordingly, the term “mobile device” or “electronic device:” as generally used herein refers to any data processor and includes Internet appliances, hand-held devices (including tablets, computers, wearable computers, cellular or mobile phones, multi-processor systems, processor-based or programmable consumer electronics, network computers, minicomputers) and the like.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “color vision test system”, “vision test system”, “TrueHue system”, and “system” are interchangeable and refer to the color vision deficiency digital test system 100 of the present invention.

Notwithstanding the forgoing, the color vision deficiency digital test system 100 of the present invention can be of any suitable configuration as is known in the art without affecting the overall concept of the invention, provided that it accomplishes the above-stated objectives. One of ordinary skill in the art will appreciate that the configuration and specification of the components of the color vision deficiency digital test system 100 as shown in FIGS. 1-9 is for illustrative purposes only, and that many other configurations of the color vision deficiency digital test system 100 are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A color vision test system for improving color vision test functionality, the color vison test comprising:
a color vision test system having a computer application accessible using an electronic device, a statistical model, a clinical information database, and a user information database;
wherein said application is downloaded and installed in said electronic device;
wherein said color vision test system having a server system including an application server for providing user interfaces to enable a plurality of users to test their color vision using said application;
wherein said statistical model integrated in said server system for interpreting test results from said color vision test system to understand color vision capabilities;
wherein said statistical model programmed to provide responses to queries asked by users of said color vision test system for providing personalized and interactive support to the users;
wherein said clinical information database stores color vision threshold data used for providing color vision diagnosis to the users and for including FDA information for correct identification of said color vision diagnosis across a spectrum;
wherein said user information database stores said color vision diagnosis of the users;
wherein said color vision test having a user interface including an interactive color representation model;
wherein said interactive color representation model collects data from the user for said color vision diagnosis;
wherein said interactive color representation model having a central color matrix, an inner matrix, and an outer matrix; and
further wherein said interactive color representation model having a black partition separating said inner matrix and said outer matrix.

2. The color vision test system of claim 1, wherein said central color matrix having a plurality of color combinations.

3. The color vision test system of claim 2, wherein for said color vision test, a color of said inner matrix is matched with a color of said outer matrix.

4. The color vision test system of claim 3, wherein during said color vision test, the user selects an interactive marker across said central color matrix such that the color of said inner matrix dynamically adjusts based on the position of the user's said interactive marker on said central color matrix.

5. The color vision test system of claim 4, wherein upon matching the color of said inner matrix to the color of said outer matrix, the user lifts up the marker to select the color.

6. The color vision test system of claim 5, wherein the color of said outer matrix remains static and the color of said inner matrix changes dynamically based upon movement of the user's said interactive marker along said central wheel.

7. The color vision test system of claim 6, wherein said user information database further stores personal information selected from a group consisting of a name, an age, a geography, a gender, and a prior said color vision diagnosis.

8. The color vision test system of claim 7, wherein said server system further having a communication network selected from a public network, an Internet, and a private network.

9. The color vision test system of claim 8, wherein said computer application having a series of prompts selected from the group consisting of a ‘Take the test’, a “Are you color deficient?”, a “Not Color Blind”, a “I Don't Know”, a “Red-Green Color Blind”, and a “Blue-Yellow Color Blind”.

10. The color vision test system of claim 9, wherein said collected data identifies color vision deficiencies.

11. The color vision test system of claim 8, wherein said communication network accessed using a website, a browser, and a uniform resource location (URL).

12. A color vision test method for improving color vision test functionality, the color vison test method comprising the following steps:
providing a color vision test system having a computer application accessible using a electronic device, a clinical information database, a user information database, and a server system, wherein said application is downloaded and installed in said electronic device, and further wherein said server system including an application server for providing user interfaces to enable a plurality of users to test their color vision using said application;
interpreting test results from said color vision test system to understand color vision capabilities;
providing responses to queries asked by users of said color vision test system for providing personalized and interactive support to the users;
storing color vision threshold data used for providing color vision diagnosis to the users in said clinical information database for correct identification of said color vision diagnosis across a spectrum;
storing said color vision diagnosis of the users in said user information database;
interfacing a user with said color vision test including an interactive color representation model, wherein said interactive color representation model having a central color matrix, an inner matrix, and an outer matrix, and further wherein said interactive color representation model having a black partition separating said inner matrix and said outer matrix;
collecting data from the user and said interactive color representation model for said color vision diagnosis; and
matching a color of said inner matrix with a color of said outer matrix for said color vision test.

13. The color vision test method of claim 12, wherein said central color matrix having a plurality of color combinations.

14. The color vision test method of claim 13, wherein during said color vision test, the user selects an interactive marker across said central color matrix such that the color of said inner matrix dynamically adjusts based on the position of the user's said interactive marker on said central color matrix.

15. The color vision test method of claim 14, wherein upon matching the color of said inner matrix to the color of said outer matrix, the user lifts up the marker to select the color.

16. The color vision test method of claim 15, wherein the color of said outer matrix remains static and the color of said inner matrix changes dynamically based upon movement of the user's said interactive marker along said central wheel.

17. A color vision test method for improving color vision test functionality, the color vison test method comprising the following steps:
providing a color vision test system having a computer application accessible using an electronic device, a clinical information database, and a server system, wherein said application is downloaded and installed in said electronic device, and further wherein said server system including an application server for providing user interfaces to enable a plurality of users to test their color vision using said application;
interpreting test results from said color vision test system to understand color vision capabilities;
providing responses to queries asked by users of said color vision test system for providing personalized and interactive support to the users;
storing color vision threshold data used for providing color vision diagnosis to the users in said clinical information database for correct identification of said color vision diagnosis across a spectrum;
interfacing a user with said color vision test including an interactive color representation model, wherein said interactive color representation model having a central color matrix, an inner matrix, and an outer matrix, and further wherein said interactive color representation model having a black partition separating said inner matrix and said outer matrix;
collecting data from the user and said interactive color representation model for said color vision diagnosis;
matching, in a series of rounds, a color of said inner matrix with a color of said outer matrix for said color vision test; and
calculating a deviation between the color of said inner matrix and the color of said outer matrix.

18. The color vision test method of claim 17, wherein said series of rounds is at least ten (10).

19. The color vision test method of claim 18, wherein during said color vision test, the user selects an interactive marker across said central color matrix such that the color of said inner matrix dynamically adjusts based on the position of the user's said interactive marker on said central color matrix, and further wherein upon matching the color of said inner matrix to the color of said outer matrix, the user lifts up the marker to select the color.

20. The color vision test method of claim 19, wherein said calculating a deviation includes traversal data selected from the group consisting of time and distance traveled by the user's said interactive marker.
